(19) United States
(12) Patent Application Publication
(10) Pub. No.: US 2018/0346950 A1
(43) Pub. Date: Dec. 6, 2018

(54) INACTIVATION OF PROTEASES

(71) Applicants: Alexander JACOBI, Laupheim (DE); Dorothee AMBROSIUS, Laupheim (DE); Philine DOBBERTHIEN, Warthausen (DE); Christian ECKERMANN, Biberach an der Riss (DE); Franz NOTHELFER, Biberach an der Riss (DE)

(72) Inventors: Alexander JACOBI, Laupheim (DE); Dorothee AMBROSIUS, Laupheim (DE); Philine DOBBERTHIEN, Warthausen (DE); Christian ECKERMANN, Biberach an der Riss (DE); Franz NOTHELFER, Biberach an der Riss (DE)

(73) Assignee: Boehringer Ingelheim International GmbH, Ingelheim am Rhein (DE)

(21) Appl. No.: 16/059,584

(22) Filed: Aug. 9, 2018

Related U.S. Application Data
(63) Continuation of application No. 13/212,486, filed on Aug. 18, 2011, now abandoned.

(30) Foreign Application Priority Data
Aug. 20, 2010 (EP) 101 73 540.5

(51) Int. Cl. C12P 21/00; C12N 9/64; C07K 1/14; C12N 9/99

(52) U.S. Cl. CPC C12P 21/00; C12N 9/64; C12N 9/99; C07K 1/14

(57) ABSTRACT

The invention relates to a process for inactivating proteases by repeatedly changing the pH in the cell culture supernatant at the start of the process for the purification of biopharmaceuticals. The pH is adjusted first to 3-5, and then to 7-9.

### BACKGROUND TO THE INVENTION

### Technical Field

The invention is in the field of the manufacture of biopharmaceutical products. It relates in particular to improving the process for preparing biopharmaceutical products by the inactivation of proteolytically active enzymes in the cell-free cell culture supernatant.

### BACKGROUND

Biomolecules such as proteins, polynucleotides, polysaccharides and the like are increasingly gaining commercial importance as medicines, as diagnostic agents, as additives to foods, detergents and the like, as research reagents and for many other applications. The need for such biomolecules, for example, in the case of proteins, can generally no longer be satisfied—by isolating molecules from natural sources, but rather requires the use ps of biotechnological production methods.

The biotechnological production of proteins typically begins with the cloning of a DNA fragment into a suitable expression vector. After transfection of the expression vector into suitable prokaryolic or eukaryotic expression cells and subsequent selection of transfected cells the latter are cultivated in fermenters and the desired protein is expressed. Then the cells or the culture supernatant is or are harvested and the protein contained therein is worked up and purified.

It is known that proteases are present in the harvested liquid, e.g. in cell-free culture supernatant. Both biopharmaceuticals such as monoclonal antibodies or recombinant proteins as well as chromatography materials such as immobilised protein A can be very rapidly degraded or structurally damaged by proteases. This leads to compromises in the product quality (homogeneity, functionality) and, in chromatographic materials, to a reduction in the binding capacity, with consequent contamination of the bound product fractions. Particularly in serum-free cultivation and in highly productive ceils the biopharmaceuticals produced are present in high relative concentrations and are thus particularly prone to proteolytic damage to the molecular structure, leading to both a reduced yield and lower product quality.

Protein damage caused by proteases may occur even at neutral pHs, but extensive protein degradation may be observed particularly when the cell-free culture supernatant has to be adjusted to acidic pH levels for the purification process, for example, in order to create the desired binding conditions for the capture step, e.g. cation exchange chromatography (conditioning).

It is known that some proteases, e.g. digestive enzymes such as pepsin, are irreversibly inactivated by changes to the pH level (Z. Bohak, Purification and Characterization of Chicken Pepsinogen and Chicken Pepsin, Journal of Biological Chemistry 244 (17) (1969) 4633-4648; B. Turk, V. Turk, Lysosomes as Suicide Bags in Cell Death: Myth or Reality?, Journal of Biological Chemistry 284 (33) (2009) 21783-21787). The addition of protease inhibitors has also been proposed (A.J. Barrett, A. A. Kembhavi, M. A. Brown. H. Kirschke, C. G. Knight, M. Tamal and K. Hanada, L-trans-Epoxysuccinyi-ieucylamldo(4-guanidino)butane (E-64) and its analogues as inhibitors of cysteine proteinases including cathepsins B. Hand L. Biochem, J. 201 (1982) 189-198), However, these are very expensive, toxic and difficult to eliminate from the product. Therefore they are not an option for the economical production of safe pharmaceutical agents.

### BRIEF SUMMARY OF THE INVENTION

The invention relates to a method of inactivating proteases by repeated change in the pH in the cell culture supernatant at the beginning of the process for the purification of biopharmaceutioal agents. Advantages of the invention are an improvement in product quality and product yield, and a longer shelf life for chromatographic materials.

Surprisingly, it was found that in the harvested cell-free fermentation supernatants of mammalian cell lines (e.g. CHO, “Chinese hamster ovary” cells), proteases are present which can be activated by a change to an acidic pH and can also be irreversibly inactivated in their activity at the optimum pH by a subsequent change the pH to the neutral range.

Proteases that are active at neutral pH values can also be irreversibly inactivated in their activity under neutral conditions by a change to acidic pH values.

The present invention particularly relates to a process for inactivating proteases in liquids which are obtained from cell cultures, comprising the steps of:
(a) adjusting the pH of the liquid to 3 to 5, and then
(b) adjusting the pH of the liquid to 7 to 9.

### BRIEF DESCRIPTION OF THE DRAWINGS

FIG. 1: Activation and inactivation of proteases from ceH culture supernatant/fluid (CCF) by changing the pH twice.

FIG. 2: Degradation of the model substrate interferon (IFN) at an acidic pH. 0.1 mg/ml of interferon was incubated at 37° C. with 10% (v/v) cell culture supernatant (CCF) at pH 4 for 0 or 14 hours with and without a change in pH (lane 2 to 4) and separated by means of SDS-PAGE.

The change in pH took place at 20° C. with holding stages of 5 minutes each at pH 4 and at pH 7. IFN is degraded significantly less by the change in pH (lane 3) than when there is no pH inactivation. After 14 hours IFN is completely degraded (lane 4).

Layout of Lanes:
1-marker
2-IFN (0.1 mg/ml) before incubation
3-CCF+IFN pH 4 with change in pH, 14 h incubation
4-CCF+IFN pH 4 without change in pH, 14 h incubation

FIG. 3: Degradation of the protein IFN by three hours' incubation with CCF, 10% (v/v) at pH 4.0, analysis with RP-HPLC. After activation of proteases by acidification of the CCF, inactivation of the proteases by neutralization and subsequent incubation with IFN at pH 4.0. After three hours, 72% of the IFN can still be detected by RP-HPLC, whereas at the same time, without inactivation, only 43% of the IFN is still intact. The proteolytic activity can thus be reduced by half compared with a wild-type protein.

FIG. 4: Fluorescence assay at an acid and neutral pH. Proteases in the CCF are active at pH 3.5 (⋅) and at pH (▴). As a measurement of the proteolytic activity produced byproteases present in the CCF at pH 3.5 and pH 7, the release of a fluorophore by cleaving the Dnp-peptide-Nma-substrate as disclosed in Example 3 was measured.

FIG. 5: Proteolytic activity of neutral proteases with and without a change in pH. Neutral proteases may be almost completely and irreversibly inactivated by acidification to pH<5 and subsequent neutralization, per the procedures described in Example 3. The measurement was carried out at pH 7 in each case, the release of a fluorophore by cleaving a peptide substrate was measured as a measurement of the proteolytic activity.

FIG. 6: Proteolytic activity of acid proteases without and with a change in pH. The activation/inactivation of the proteases in the CCF was carried out by changing the pH analogously to FIG. 1, per the procedures described in Example 3. Activated proteases are active at pH<5 and cleave the substrate. Activated proteases which had been inactivated by a brief incubation at pH≥7 exhibit a residual activity reduced to 35% at pH 3.7. The measurement was carried out at pH 3.7 in each case, the release of a fluorophore by cleaving a peptide substrate was measured as an indication of the proteolytic activity.

### DETAILED DESCRIPTION OF THE INVENTION

The invention relates to a process for inactivating proteases by a repeated change in the pH in the cell culture supernatant at the beginning of the purification process. Advantages of the invention are an improvement in the product quality and yieid as well as extending the shelf life of chromatographic materials.

Surprisingly, it was found that in the harvested cell-free fermentation supernatants of mammalian cell lines (e.g. CHG, “Chinese hamster ovary” cells), proteases are present which can be activated by a change to an acidic pH and can also be irreversibly inactivated in their activity at the optimum pH by subsequently shift of the pH to the neutral range.

Proteases that are active at neutral pH values can also be irreversibly inactivated in their activity under neutral conditions by a change to acidic pH values.

In another aspect the invention relates to a process for reducing protein degradation in liquids which are obtained from cell cultures, comprising the steps of:
(c) adjusting the pH of the liquid to 3 to 5, and then
(d) adjusting the pH of the liquid to 7 to 9.

After acidification of the cell culture supernatant, there is obviously activation of the proteases that are obtained, which indicates the presence of originally lysosomal proteolytic enzymes (cathepsins). These proteases are involved in the degradation of endocytic proteins, are ubiquitously expressed in all tissues as non-active proforms and are located intracellular in endosomes. The maturation of these compartments to form lysosomes is accompanied by a dramatic lowering of the pH which leads to both autocatalytic and in trans activation of the lysosomal proteases. The secretion of cathepsins into the extracellular space is discussed chiefly in the context of the metastatic spread of tumor tissue, and for some individual cathepsins, secretion in cell culture has also been described.

The proteolytic activity at neutral pH values can be attributed on the one hand to secreted proteases and on the other hand to proteases originally located in the membrane, which are presumably separated from the cell membrane during the production process and are further active in solution.

Typically, in biopharmaceutical processes, cells are separated from the product-containing cell culture supernatant by centrifugation or filtration. The cell-free supernatant is then sterilize by filtration (max. 0.2 μMol pore size) and diafiltered for rebuffering before the capture step. The inactivation by changing the pH twice can be carried out at the earliest immediately after the separation of the cell culture supernatant from the cells and used in any other subsequent process step.

When selecting the pH levels, the product molecule and the industrial equipment should not be damaged, and therefore pH levels<3 should be avoided (chemical modification of the product protein and increased corrosion of steel containers), as well as pH>9 (deamidation of asparagine and glutamine). The holding times at the respective pH values also depend on the stability of the product protein. The time span for the activation of proteases by acidification should be as short as possible, but advantageously at least 5 minutes (min). For example, holding times between 5 and 30 minutes are advantageous, preferably 5 to 15 minutes. With longer holding times for activation at acidic pH levels, there may be increased proteolytic degradation of the target protein. The time span of the subsequent holding step for inactivation of the acid proteases at a neutral pH is not critical and the pH can also be maintained over several process steps or varied again, as all neutrally active proteases have already been inactivated and no more proteolytic activity can be detected. Advantageous holding times for the neutralizing step are 5 to 80 minutes, for example.

The adjustment to the respective target pH values may be carried out in solution by a one-time addition or titration of acids such as acetic acid or hydrochloric acid or lyes/bases such as sodium hydroxide solution or Tris, with stirring. In the acid step pH values of between 3 and 5 are advantageously selected, for example a pH of 3.5 to 4.5, preferably 4. For the subsequent inactivation of acid proteases by neutralization, pH values of 7-9 have proved effective, preferably pH 7.4-8.5.

The invention may be carried out in a temperature range of 4° C.-37° C., preferably 15° C. to 37° C., preferably 20-37° C. A preferred range for performing the invention is 20° C. to 30° C.

The process tor inactivating acid and neutral proteases by changing the pH twice was successfully carried out on cell-free culture supernatants of mammalian cells (CHO and NSO). The results can also be transferred to culture supernatants of other production organisms and can be used within the scope of the requirements of the product protein, particularly its pH stability, in the manufacture of various biopharmaceutical products.

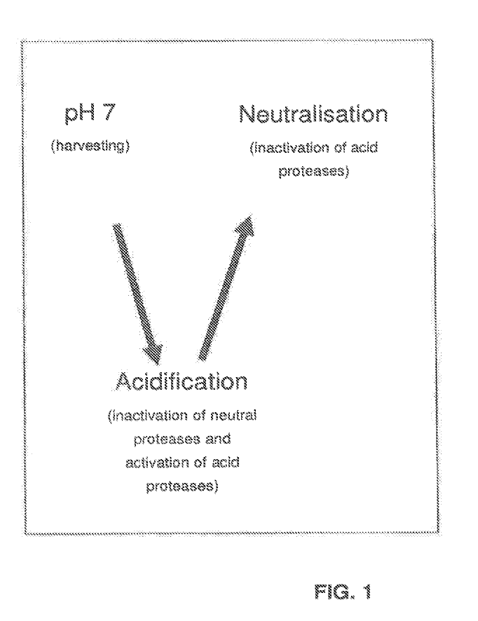

The present invention employs purely physico-and biochemical methods. By changing the pH twice through different pH units (FIG. 1) up to 75% of the acid protease activity and up to 90% of the neutral protease activity can be irreversibly eliminated. The protease activity can be detected using two defection methods, a) by the release of fluorescence after peptide cleavage and b) by the degradation of native protein substrates.

Proteases that are harmful to the product and equipment can be irreversibly inactivated during the production of biopharmaceutical medications by a quick and simple physicochemical method. The cell culture supernatant can be used in a variable manner as a result and may be obtained by a variety of purification techniques.

The addition of acids and lyes to tanks is very quick and simple to perform and can also be scaled up for industrial use. After the inactivation of the proteases the cell culture supernatant may be adapted to the purification processes in a very variable manner. Holding times or pH values are non-critical, by contrast with the conventional production processes.

### EXAMPLES

Working Example 1: Change in pH After Harvesting, Before the Capture Step

CHO cells are cultivated over 11 days in the fed batch, final volume 80 L for 11 days. The cell culture supernatant (CCF) is maintained at 20° C. and separated from the cells using a throughflow disc centrifuge and sterilized by filtration through a filter cascade. Then the pH value of the CCF is lowered to pH 4 by the addition of acetic acid. After the target pH has been reached it is maintained for 5-10 min, before the CCF is neutralized to pH 7.5 by the addition of sodium hydroxide solution. Before the additional processing (capture step) the CCF is ultra/diafiltered through a 50 kD MWCO membrane in order to achieve suitable binding conditions for the capture step.

Working Example 2: Change in pH Directly After the Separators of CCF and Cells, Before Sterile Filtration

CHO cells are cultivated over 11 days in the fed batch, final volume 80 L. The cell culture supernatant (CCF) is separated from the cells using a throughflow disc centrifuge and maintained at 20° C. Then the pH value of the CCF is lowered to pH 4 by the addition of acetic acid with constant stirring. After the target pH has been reached it is maintained for 5-10 min. before the CCF is neutralized to pH 7.5 by the addition of sodium hydroxide solution. The treated CCF is then sterilized by filtration through a filter cascade. Before the additional processing (capture step) the CCF is ultra/diafiltered through a 50 kD MWCO membrane in order to achieve suitable binding conditions for the capture step.

Working Example 3: Change in pH After rProtA Capture Step, Before Inactivation of the Virus

CHO cells are cultivated for 11 days in the fed batch, final volume 80 L. The cell culture supernatant (CCF) is separated from the cells using a throughflow disc centrifuge, sterilized by filtration through a filter cascade and ultra-diafiltered through a 50 kD MWCO membrane in order to achieve suitable binding conditions for the capture step, rProteinA affinity chromatography on PBS pH 7.5. A MabSelect chromatography column is charged with 32 mg of mAb per ml of column matrix and the antibody is eluted in a step with acetatebuffer pH 3.5. The pH of the fraction containing the product is adjusted to pH 7.5 by the addition of 1 M Tris, with stirring, and the pH is maintained for 10 min at room temperature before suitable conditions for an acidic inactivation of the virus are selected.

### Material and Methods

Cell Culture Supernatant

Murine and CHO production cell lines optimized to the secretory production of therapeutic proteins are cultivated for several days in serum-free medium. The cell culture supernatant (CCF, cell free cell culture fluid) is separated by filtration or centrifugation from cells and insoluble constituents and after being adjusted to the respective pH it is used at 10-20% (v/v) for the activity assays.

Adjustment of the pH Value

The cell culture supernatants are acidified by the addition of acetic acid. The samples are immediately mixed and incubated for 5-10 minutes at the selected pH. Precipitating constituents are pelleted by centrifugation and discarded. The pH is raised by the addition of sodium hydroxide solution or 1 M Tris base.

Inhibition Experiments for Determining the Protease Classes

In order to inhibit individual protease classes, CCF is incubated with different commercial inhibitors and any remaining activity is then investigated in the activity assays. The concentration of inhibitor used is according to the manufacturer's recommendations.

### Activity Assays

Florescence Assay, Analysis By the Release of a Fluorophore

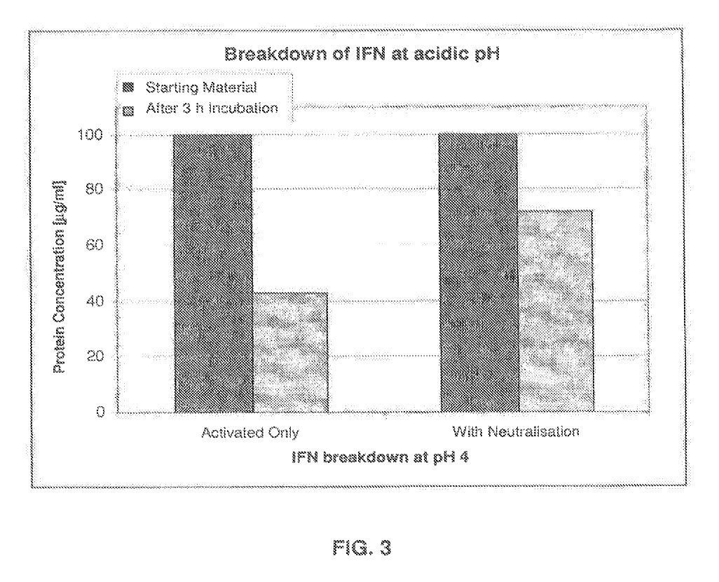

The substrates used for the kinetic and quantitative determination of the proteolytic, activity are various peptide-fluorophore conjugates, whose cleavage leads to the release of fluorescent dyes such as aminomethylcoumarin (AMC) or the elimination of the quenching effect by dinitrophenyl (Dnp) on N-methylamninobenzoyl-diaminopropionic acid (Nma). The increase in the fluorescence signal may be monitored photometrically at λEx=380 nm; λEm=460 nm (AMC), λEx=340 nm; λEm=460 nm in the Multilabel-Counter Victor^{2,3} (Perkin Elmer, Mass., USA) (FIG. 3).

All the assays for kinetic and quantitative analyses are performed in the substrate saturation at 0.2 mM Peptide-AMC, 10-20 M Peptide-MCA or 5-10 M DnP-Peptide-Nma in the presence of 10-20% (v/v) CCF at pH 7 (100 mM Tris/HCI, 200 mM NaCl) and pH 3.5 (100 mM Na-acetate, 200 mM NaCl).

Degradation Assay, Analysis by PAGE and RP-HPLC

The substrate used for the qualitative analysis of proteolytic activity is a native protein of the interferon family (IFN). IFN is 22.5 kD in size and is present as a monomer in solution.

For the degradation assay, 0.1 mg/milFN are incubated with 10% (v/v) CGF at 37° C. at pH 4 for up to 24 hours and detected by SDS-Page and silver staining according to Heukeshoven (Heukeshoven, J., Dernick, R., Improved silver staining procedure for fast staining in PhastSystem Development Unit, I. Staining of sodium dodecyl sulphate gels. Electrophoresis, 9, (1988) 28-32.) or the degradation of the protein is determined by RP-HPLC as a measurement of proteolytic activity.

The RP-HPLC analysis is carried out on an HPLC apparatus made by Waters (Waters 2695 alliance) with a UV detector (Waters 2487 Dual Absorbance Detector) by means of a Vydac 214 TP-C₄ column by gradient elution of 0.2% (v/v) TFA in water (solution A) to 0.15% (v/v) TFA in acetonitrile (solution B) (Table 1).

TABLE 1
Elution gradient for a RP-HPLC-C4 column. The gradient
runs from aqueous to organic solvent

| time | solution A | solution B |
|---|---|---|
| [min] | [%] | [%] |
| 5.0 | 60 | 40 |
| 15.0 | 30 | 70 |
| 15.1 | 10 | 90 |
| 19.0 | 10 | 90 |
| 19.1 | 60 | 40 |
| 21.0 | 60 | 40 |

### RESULTS

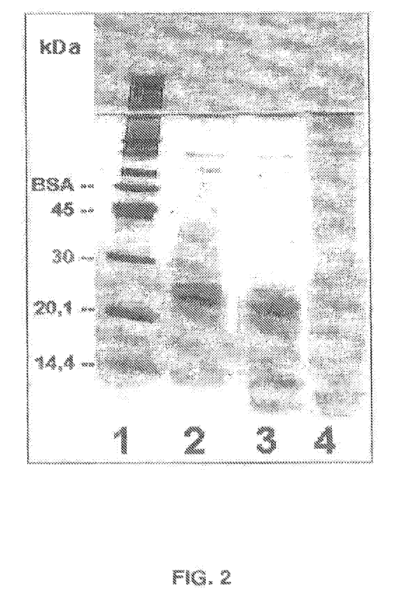

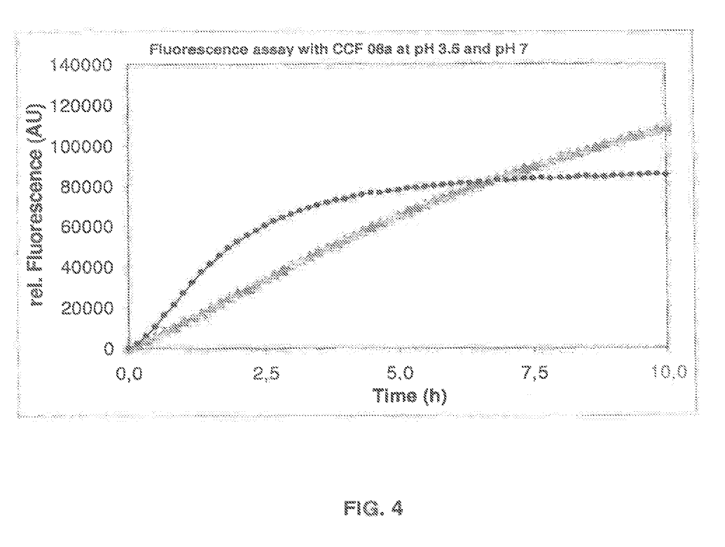

The substrate cleavage by proteases from CCF has two peaks which are situated at acidic pH values pH<5 and in the neutral range around pH 7. The activities of the respective proteases may be monitored by protein degradation and the release of fluorescence after the cleaving of a fluorogenic peptide substrate (FIG. 2 and 4). FIG. 2 shows the degradation of IFN at pH 4 in the presence of 10% (v/v) CCF. After only a few hours IFN is degraded under acidic conditions (lane 4). FIG. 4 shows the course of the cleavage over time of the Dnp-peptide-Nma-substrate by 20% (v/v) CCF after incubation at pH 3.5 and pH 7. The increasing fluorescence signal is a measurement of the cleavage of the peptide substrate. Activity is observed at both acidic and neutral pH values.

This activity can be suppressed by protease class-specific inhibitors, thereby showing that several protease classes are present in the CCF and that the latter can be divided into two groups: the acidically-active proteases which are active only at low pH values, and the neutrally-active proteases which are active only at neutral pH values. The acidically active ones have no activity at neutral pH values and the neutrally active ones have no activity at acidic pH values. While the neutrally-active proteases are already active at the time of cell separation, the activation of the acidically-active proteases in CCF does not take place until the reaction conditions are acidified.

Two-Step Change in the pH in Order to Inactivate Neutral Proteases

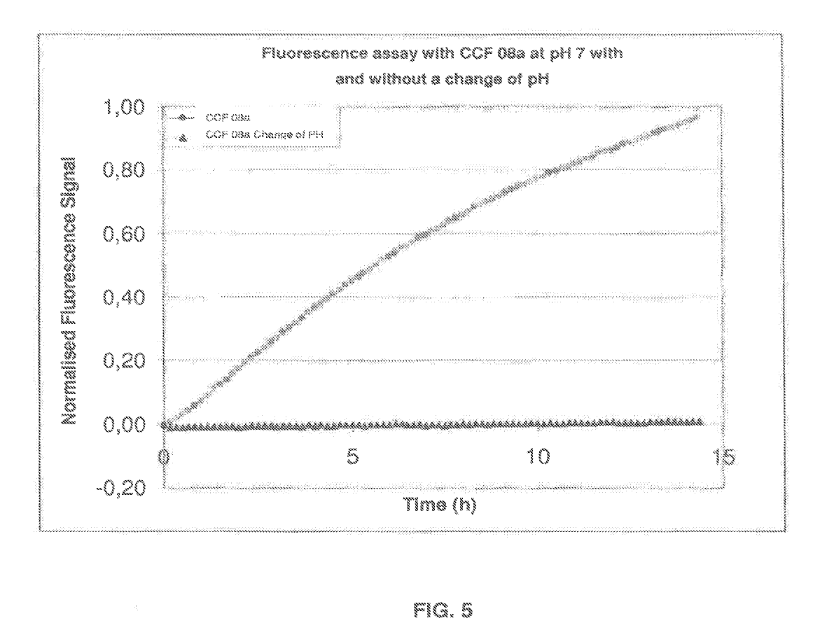

The activity of neutral proteases from untreated CCF may be determined at pH 7 in the fluorescence assay (FIG. 5, circles). If the CCF is subjected to a two-step change in pH from pH 7 to pH<5 followed by neutralization, virtually no further activity can be detected in the fluorescence assay at pH 7 (FIG. 5, triangles).

The brief acidification of the CCF and subsequent neutralization results in total and irreversible loss of the proteolytic activity of neutrally-active proteases. p

### Two-Step Change in the pH in Order to Inactivate Acidic Proteases

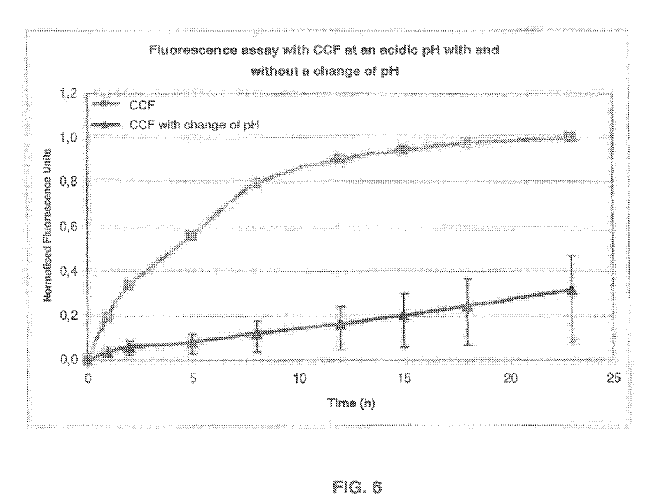

The proteolytic activity of the proteases activated by the acidification of the CCF may be detected at pH 3.5 in the fluorescence assay and in the protein degradation assay (FIG. 6, squares). However, this activity is not maintained if the CCF is neutralized after the acidification. If the reaction conditions of pH 3.5 that are optimal for acidic proteases are restored after the neutralization, the activity of the acidic proteases that is measurable in the fluorescence assay is reduced by up to 65% relative to the untreated CCF (FIG. 6, triangles).

The degradation of proteins is also reduced by the double change in the pH values. The degradation of the model protein IFN is significantly reduced after the neutralization step (FIG. 3, grey bars).

It was possible to reduce the degradation of the native protein substrate IFN by 50% as a result of the repeated change in the pH (FIG. 3, quantification by RP-HPLC).

1. A process for inactivating proteases in a liquid that is obtained from a cell culture, comprising the steps of:
(a) adjusting the pH of the liquid to 3 to 5, and then
(b) adjusting the pH of the liquid to 7 to 9.

2. A process for reducing the protein degradation in a liquid that is obtained from a cell culture, comprising the steps of
(a) adjusting the pH of the liquid to 3 to 5, and then
(b) adjusting the pH of the liquid to 7 to 9.

3. A process according to claim 1 or 2, characterised in that the pH in step (a) is in the range from 3.5 to 4.5.

4. A process according to claim 1 or 2, characterised in that the pH in step (a) is maintained for a period of 5 minutes to 30 minutes.

5. A process according to claim 1 or 2, characterised in that the pH in step (a) is carried out at a temperature of 20 to 30° C.

6. A process according to claim 1 or 2, characterised in that the pH in step (b) is in the range from 7.4 to 8.5.

7. A process according to claim 1 or 2, characterised in that the pH in step (b) is maintained for a period of 5 minutes to 60 minutes.

8. A process according to claim 1 or 2, characterised in that the pH in step (b) is carried out at a temperature of 20 to 30° C.

9. A process according to claim 1 or 2, characterised in that the liquid is liquid from a mammalian cell culture.

10. A process according to claim 1 or 2, characterised in that the liquid is cell-free liquid from a mammalian cell culture.
